(19) United States
(12) Patent Application Publication
(10) Pub. No.: US 2023/0097789 A1
(43) Pub. Date: Mar. 30, 2023

(54) INHIBITORS OF EGFR, KRAS, BRAF, AND OTHER TARGETS AND USE OF THE SAME

(71) Applicant: THE REGENTS OF THE UNIVERSITY OF MICHIGAN, Ann Arbor, MI (US)

(72) Inventors: Mukesh K. Nyati, Superior Township, MI (US); Theodore S. Lawrence, Ann Arbor, MI (US); Christopher Whitehead, Ann Arbor, MI (US); Jason Christopher Rech, Ann Arbor, MI (US); Brennan Taylor Watch, Ann Arbor, MI (US); Alexander Bridges, Saline, MI (US)

(21) Appl. No.: 17/801,327

(22) PCT Filed: Mar. 5, 2021
(86) PCT No.: PCT/US21/20987

§ 371 Date: Aug. 22, 2022

Related U.S. Application Data
(60) Provisional application No. 62/985,549, filed on Mar. 5, 2020.

(51) Int. Cl. C07D 233/64; A61P 35/00; C07D 401/12

(52) U.S. Cl. CPC C07D 233/64; A61P 35/00; C07D 401/12

(57) ABSTRACT

Provided herein are compounds that are useful in treating cancer.

### BACKGROUND

The EGFR small molecule tyrosine kinase inhibitors (TKI's) erlotinib, gefitinib, and afatinib have been most successful as single agents in the treatment of lung adenocarcinomas that have somatic mutations (such as L858R or deletion in exon 19, i.e.

E746-A750) that confer sensitivity to this class of drugs, which occur in 7-20% of patients depending on ethnicity and gender (19). Unfortunately, responses rarely last more than a year because virtually all patients develop resistance to therapy (20). A third-generation irreversible inhibitor, osimertinib (AZD9291), is effective in treating naïve as well as patients who have acquired resistance to first or second generation TKIs (7). However, within a year of treatment with osimertinib, a majority of patients develop another mutation in the EGFR kinase domain (C797S), which is the drug binding site (12, 21, 22). Although several approaches to target osimertinib resistant EGFR have been reported (12, 13, 23), as of now no TKI treatment option exists for these patients with this C797S mutation. Chemotherapy is the only option.

The RAS family is comprised of three members, KRAS, NRAS, and HRAS. KRAS is the single most frequently mutated oncogene in human cancers. KRAS mutations are prevalent in the cancerous cells of patients having any one of the three most refractory cancer types in the United States: 95% of pancreatic cancers, 45% of colorectal cancers, and 35% of lung cancers.

Because of the prevalence of KRAS mutations in particularly intractable cancers, intensive drug discovery efforts have been devoted to developing therapeutic strategies that block KRAS function. These efforts include (i) direct targeting approaches, such as disrupting protein-protein (e.g., RAS-Raf) interactions and covalent irreversible KRAS-G12C inhibition; and (ii) indirect targeting approaches, such as decreasing the RAS population at the plasma membrane and targeting downstream effector signaling proteins (e.g., ERK or mTOR). Despite extensive efforts, a clinically viable cancer therapy that effectively blocks KRAS function has remained elusive.

In view of the foregoing, there exists a need for a cancer therapeutic that targets EGFR, KRAS, cMET, BRAF, and/or other target. There also exists a need for a therapeutic that treats cancer without drug resistance developing after initial use.

### SUMMARY

Provided herein are compounds and methods for treating cancer. More particularly, provided are modulators of EGFR, KRAS, and/or BRAF, and the uses of such modulators in treating or preventing diseases or disorders associated with aberrant activity of those targets, e.g., cancer.

The disclosure provides compounds, or pharmaceutically acceptable salts thereof, of Formula I:

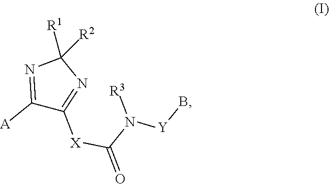

wherein X is C₁₋₆ alkylene, C₂₋₆ alkenylene, C₂₋₆ alkynylene, C₃₋₁₀ cycloalkylene, 4-6 membered heterocycle, O—C₀₋₆alkylene, O—C₂₋₆ alkenylene, O—C₂₋₆ alkynylene, O—C₃₋₁₀ cycloalkylene, O-(4-6 membered heterocyclene), S—C₀₋₆alkylene, S—C₂₋₆ alkenylene, S—C₂₋₆ alkynylene, S—C₃₋₁₀cycloalkylene, S-(4-6 membered heterocyclene), NR³—C₀₋₆alkylene, NR³—C₂₋₆alkenylene, NR³—C₂₋₆alkynylene, NR³—C₃₋₁₀ cycloalkylene, or NR³-(4-6 membered heterocyclene), and X is optionally substituted with 1-5 groups independently selected from R³;
Y is C₀₋₆alkylene, C₃₋₆alkenylene, or C₃₋₆alkynylene, and Y is optionally substituted with 1-3 groups independently selected from halo, N(R³)₂, and R³;
A is C₆₋₁₀ aryl or 5-10 membered heteroaryl having 1-4 heteroatoms selected from N, O, and S, and A is optionally substituted with 1 to 3 R⁴;
B is C₆₋₁₀ aryl, 5-10 membered heteroaryl having 1-4 heteroatoms selected from N, O, and S, 3-8 membered cycloalkyl ring, or a 4-10 membered heterocycle having 1-3 heteroatoms selected from N, O, and S, and B is optionally substituted with 1 to 3 R⁵;
R¹ and R² are each independently C₁₋₆ alkyl, C₃₋₆ alkenyl, C₃₋₆ alkynyl, or C₃₋₆ cycloalkyl, or R¹ and R² together with the carbon atom to which they are attached form a 4-8 membered cycloalkyl or heterocycle, wherein the heterocycle has 1 or 2 ring heteroatoms selected from O, S, and N, and wherein said cycloalkyl or heterocycle is optionally substituted with 1-2 R⁴;
each R³ is independently OH, C₁₋₆ alkyl, C₂₋₆alkenyl, C₂₋₆alkynyl, C₁₋₆alkoxy, phenyl, O-phenyl, benzyl, O-benzyl, C₃₋₆cycloalkyl, 4-10 membered heterocycle having 1 to 4 heteroatoms selected from N, O, and S, or (O)₀₋₁-5-10 membered heteroaryl having 1 to 3 heteroatoms selected from N, O, and S, or two R³ taken together with the atom(s) to which they are attached form a C₃₋₆ cycloalkyl (e.g., C₄₋₆ cycloalkenyl), or 4-6 membered heterocycle having one heteroatom selected from N, O and S;
each R⁴ and R⁵ is independently halo, NO₂, oxo, cyano, C₁₋₄ alkyl, C₁₋₄haloalkyl (e.g., CF₃, CHF₂), C₁₋₄alkoxy, C₁₋₄haloalkoxy (e.g., OCF₃, OCHF₂), C₁₋₄thioalkoxy, C₂₋₄alkenyl, C₂₋₄alkynyl, CHO, C(═O)Rs, C(═O)N(R⁶)₂, S(O)₀₋₂R⁶, SO₂N(R⁶)₂, NH₂, NHR⁶, N(R⁶)₂, NR⁷COR⁶, NR⁷SO₂R⁶, P(═O)(R⁶)₂, C₃₋₆cycloalkyl, 4-10 membered heterocycle having 1 to 4 heteroatoms selected from N, O, and S (e.g., oxetanyl, oxetanyloxy, oxetanylamino, oxolanyl, oxolanyloxy, oxolanylamino, oxanyl oxanyloxy, oxanylamino, oxepanyl, oxepanyloxy, oxepanylamino, azetidinyl, azetidinyloxy, azetidylamino, pyrrolidinyl, pyrolidinyloxy, pyrrolidinylamino, piperidinyl, piperidinyloxy, piperidinylamino, azepanyl, azepanyloxy, azepanylamino, dioxolanyl, dioxanyl, morpholino, thiomorpholino, thiomorpholino-S,S-dioxide, piperazinyl, dioxepanyl, dioxepanyloxy, dioxepanylamino, oxazepanyl, oxazepanyloxy, oxazepanylamino, diazepanyl, diazepanyloxy, or diazepanylamino);
each R⁶ is independently H, C₁₋₆ alkyl, C₁₋₆ haloalkyl, C₃₋₆ alkenyl, C₃₋₆ alkynyl, COOR⁷, CON(R⁷)₂, C₀₋₃alkylene-C₃₋₈cycloalkyl, C₀₋₃alkylene-C₆₋₁₀aryl, C₀₋₃alkylene-(4-10 membered heterocycle having 1-4 heteroatoms selected from N, O, and S), or C₀₋₃alkylene-(5-10 membered heteroaryl having 1-4 heteroatoms selected from N, O, and S), wherein the aryl, heterocyle, or heteroaryl is optionally substituted with 1 to 3 R⁷; and
each R⁷ is independently H, C₁₋₆ alkyl, C₁₋₆ haloalkyl, C₃₋₆ alkenyl, C₃₋₆ alkynyl, C₁₋₄alkoxy, or C₁₋₄haloalkoxy,
with the proviso that the compound is not a compound (or salt thereof) as disclosed in WO 2019/165358.

Further provided herein are methods of using the compounds disclosed to modulate EGFR, KRAS, cMET, and/or BRAF. Other aspects of the disclosure include methods of using the compounds disclosed to inhibit EGFR dimerization, and methods of using the compounds disclosed to induce EGFR degradation. In some cases, the methods include using the compounds disclosed herein to modulate KRAS. In some cases, the methods include using the compounds disclosed herein to modulate cMET. In some cases, the methods include using the compounds disclosed herein to modulate BRAF.

Other aspects of the disclosure include a compound as disclosed herein for use in the preparation of a medicament for treating or preventing a disease or disorder associated with aberrant activity of EGFR, KRAS, cMET, and/or BRAF in a subject,

### DETAILED DESCRIPTION

Although inhibition of the kinase activities of oncogenic proteins using small molecules and antibodies has been a mainstay of anticancer drug development efforts, resulting in several FDA-approved cancer therapies, the clinical effectiveness of kinase-targeted agents has been inconsistent. EGFR has been shown to exhibit scaffold functions in addition to its tyrosine kinase activity. This is demonstrated by either expressing a kinase-dead (KD) mutant of EGFR (e.g. K745A, V741G, and Y740F) or by expressing ErbB3 (which has no kinase activity) in Ba/F3 cells that do not express these receptors. Expression of these kinase-defective mutants promotes cell survival, indicating that these receptors can still transmit a survival signal perhaps by forming dimers, suggesting that EGFR has functions beyond kinase activity.

EGFR dimers are known to be relatively stable when compared to the monomers. Dimers are capable of generating downstream mitogenic signaling. Without being bound by theory, it is hypothesized that blocking EGFR dimerization would accelerate degradation of EGFR, and that this approach would be effective against tumors that are driven by TKI resistant EGFR. Briefly, it was demonstrated that EGF bound EGFR (that is phosphorylated-EGFR, prevalent in most tumors) protein stability is regulated by formation of dimers via a segment within the kinase domain of EGFR that lies between αC helix and β4 sheets of the c-lobe and h-helix of the n-lobe of the EGFR kinase domain. EGFR protein stability in normal cells is not primarily regulated by this dimer interface because, in the absence of EGF, EGFR does not form an asymmetric dimer. This difference between tumor and normal cells provides a new targetable protein-protein interaction.

To test this idea, over a dozen peptides that mimic this binding surface were generated. The most effective peptide, containing the six amino acids from the αC-β4 loop of the EGFR, was named Disruptin. Disruptin is capable of inhibiting EGF-induced dimerization of EGFR. This peptide binds directly to EGFR, and this binding is not affected significantly with repeated HEPES washes compared to a control (scrambled) peptide. Although Disruptin is effective in a tyrosine kinase inhibitor (TKI) resistant lung xenograft model, delivery of peptides in humans remains challenging.

Provided herein are compounds which modulate EGFR, for example, compounds which block EGFR dimerization, induce EGFR degradation, and kill EGFR driven cells. These compounds are useful in the prevention or treatment of a variety of diseases and disorders, for example, in the treatment of cancer.

As such, provided herein are compounds, or pharmaceutically acceptable salts thereof, having the structure of Formula I:

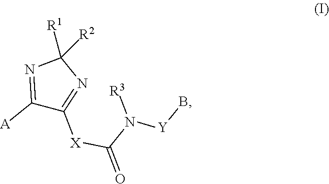

wherein X is C₁₋₆ alkylene, C₂₋₆ alkenylene, C₂₋₆ alkynylene, C₃₋₁₀ cycloalkylene, 4-6 membered heterocycle, O—C₀₋₆alkylene, O—C₂₋₆ alkenylene, O—C₂₋₆ alkynylene, O—C₃₋₁₀ cycloalkylene, O-(4-6 membered heterocyclene), S—C₀₋₆alkylene, S—C₂₋₆ alkenylene, S—C₂₋₆ alkynylene, S—C₃₋₁₀ cycloalkylene, S-(4-6 membered heterocyclene), NR³—C₀₋₆alkylene, NR³—C₂₋₆alkenylene, NR³—C₂₋₆alkynylene, NR³—C₃₋₁₀ cycloalkylene, or NR³-(4-6 membered heterocyclene), and X is optionally substituted with 1-5 groups independently selected from R³;
Y is C₀₋₆alkylene, C₃₋₆alkenylene, or C₃₋₆alkynylene, and Y is optionally substituted with 1-3 groups independently selected from halo, N(R³)₂, and R³;
A is C₆₋₁₀ aryl or 5-10 membered heteroaryl having 1-4 heteroatoms selected from N, O, and S, and A is optionally substituted with 1 to 3 R⁴;
B is C₆₋₁₀ aryl, 5-10 membered heteroaryl having 1-4 heteroatoms selected from N, O, and S, 3-8 membered cycloalkyl ring, or a 4-10 membered heterocycle having 1-3 heteroatoms selected from N, O, and S, and B is optionally substituted with 1 to 3 R⁵;
R¹ and R² are each independently C₁₋₆ alkyl, C₃₋₆ alkenyl, C₃₋₆ alkynyl, or C₃₋₆ cycloalkyl, or R¹ and R² together with the carbon atom to which they are attached form a 4-8 membered cycloalkyl or heterocycle, wherein the heterocycle has 1 or 2 ring heteroatoms selected from O, S, and N, and wherein said cycloalkyl or heterocycle is optionally substituted with 1-2 R⁴; each R³ is independently OH, C₁₋₆ alkyl, C₂₋₆alkenyl, C₂₋₆alkynyl, C₁₋₆alkoxy, phenyl, O-phenyl, benzyl, O-benzyl, C₃₋₆cycloalkyl, 4-10 membered heterocycle having 1 to 4 heteroatoms selected from N, O, and S, or (O)₀₋₁-5-10 membered heteroaryl having 1 to 3 heteroatoms selected from N, O, and S, or two R³ taken together with the atom(s) to which they are attached form a C₃₋₆ cycloalkyl (e.g., C₄₋₆ cycloalkenyl), or 4-6 membered heterocycle having one heteroatom selected from N, O and S;
each R⁴ and R⁵ is independently halo, NO₂, oxo, cyano, C₁₋₄ alkyl, C₁₋₄haloalkyl (e.g., CF₃, CHF₂), C₁₋₄alkoxy, C₁₋₄haloalkoxy (e.g., OCF₃, OCHF₂), C₁₋₄thioalkoxy, C₂₋₄alkenyl, C₂₋₄alkynyl, CHO, C(═O)R⁶, C(═O)N(R⁶)₂, S(O)₀₋₂R⁶, SO₂N(R⁶)₂, NH₂, NHR⁶, N(R⁶)₂, NR⁷COR⁶, NR⁷SO₂R⁶, P(═O)(R⁶)₂, C₃₋₆cycloalkyl, 4-10 membered heterocycle having 1 to 4 heteroatoms selected from N, O, and S (e.g., oxetanyl, oxetanyloxy, oxetanylamino, oxolanyl, oxolanyloxy, oxolanylamino, oxanyl oxanyloxy, oxanylamino, oxepanyl, oxepanyloxy, oxepanylamino, azetidinyl, azetidinyloxy, azetidylamino, pyrrolidinyl, pyrolidinyloxy, pyrrolidinylamino, piperidinyl, piperidinyloxy, piperidinylamino, azepanyl, azepanyloxy, azepanylamino, dioxolanyl, dioxanyl, morpholino, thiomorpholino, thiomorpholino-S,S-dioxide, piperazinyl, dioxepanyl, dioxepanyloxy, dioxepanylamino, oxazepanyl, oxazepanyloxy, oxazepanylamino, diazepanyl, diazepanyloxy, or diazepanylamino);
each R⁶ is independently H, C₁₋₆ alkyl, C₁₋₆ haloalkyl, C₃₋₆ alkenyl, C₃₋₆ alkynyl, COOR⁷, CON(R⁷)₂, C₀₋₃alkylene-C₃₋₈cycloalkyl, C₀₋₃alkylene-C₆₋₁₀aryl, C₀₋₃alkylene-(4-10 membered heterocycle having 1-4 heteroatoms selected from N, O, and S), or C₀₋₃alkylene-(5-10 membered heteroaryl having 1-4 heteroatoms selected from N, O, and S), wherein the aryl, heterocyle, or heteroaryl is optionally substituted with 1 to 3 R⁷; and
each R⁷ is independently H, C₁₋₆ alkyl, C₁₋₆ haloalkyl, C₃₋₆ alkenyl, C₃₋₆ alkynyl, C₁₋₄alkoxy, or C₁₋₄haloalkoxy,
with the proviso that the compound is not a compound (or salt thereof) as disclosed in WO 2019/165358.

In various embodiments, R¹ and R² are each independently C₁₋₆ alkyl. In some embodiments, R¹ and R² are each methyl.

In various embodiments, R¹ and R² together with the carbon atom to which they are attached form a 4-8 membered cycloalkyl or heterocycle. In some embodiments, R¹ and R² together with the carbon atom to which they are attached form a 5 or 6 membered cycloalkyl or heterocycle. In some embodiments, R¹ and R² together with the carbon atom to which they are attached form a cyclohexyl ring.

In various embodiments, R¹ and R² together with the carbon atom to which they are attached form a heterocycle having the structure:

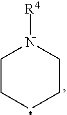

where * indicates the point of attachment to the rest of the compound of Formula I. In some embodiments, R⁴ is C₁₋₆ alkyl, C₁₋₆ haloalkyl, (C═O)R³, (C═O)OR³, CON(R³)₂, C₀₋₃alkylene-C₃₋₈cycloalkyl, C₀₋₃alkylene-C₆₋₁₀aryl, or C₀₋₃alkylene-(5-10 membered heteroaryl having 1-4 heteroatoms selected from N, O, and S), wherein the aryl or heteroaryl is optionally substituted with 1 to 3 R⁵. In some embodiments, R⁴ is C₁₋₆ alkyl, (C═O)R³, (C═O)OR³, or CON(R³)₂. In some embodiments, R⁴ is C₁₋₆ alkyl. In some embodiments, R⁴ is methyl, ethyl, propyl, isopropyl, isobutyl, or isopentyl. In some embodiments, R⁴ is methyl. In some embodiments, R⁴ is deuterated. In some embodiments, R⁴ is C₁₋₆ haloalkyl. In some embodiments, R⁴ is 3,3,3-trifluoropropyl. In some embodiments, R⁴ is C₀₋₃alkylene-C₃₋₈cycloalkyl. In some embodiments, R⁴ is cyclobutyl, cyclopentyl, or cyclohexyl. In some embodiments, R⁴ is cyclobutyl or cyclopentyl. In some embodiments, R⁴ is C₀₋₃alkylene-C₆₋₁₀aryl. In some embodiments, R⁴ is benzyl. In some embodiments, R⁴ is C₀₋₃alkylene-(5-10 membered heteroaryl having 1-4 heteroatoms selected from N, O, and S), wherein the heteroaryl is optionally substituted with 1 to 3 R⁵. In some embodiments, R⁴ is C₁alkylene-(5-10 membered heteroaryl having 1-4 heteroatoms selected from N, O, and S), wherein the heteroaryl is optionally substituted with 1 to 3 R⁵. In some embodiments, R⁴ is C₀₋₃alkylene-(5-10 membered heteroaryl having 1-4 heteroatoms selected from N, O, and S), wherein the heteroaryl is substituted with 1 to 3 R⁵. In some embodiments, R⁴ is C₀₋₃alkylene-(5-10 membered heteroaryl having 1-4 heteroatoms selected from N, O, and S), wherein the heteroaryl is unsubstituted. In some embodiments, R⁴ is

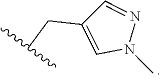 [SMILES: *Cc1cnn(C)c1]

In various embodiments, A is C₆₋₁₀ aryl. In some embodiments, A is phenyl.

In various embodiments, B is C₆₋₁₀ aryl. In some embodiments, B is phenyl. In various embodiments, B is 5-10 membered heteroaryl having 1-4 heteroatoms selected from N, O, and S. In some embodiments, B is pyridinyl. In some embodiments, B is quinolinyl. In various embodiments, B is 3-8 membered cycloalkyl. In some embodiments, B is 5 or 6 membered cycloalkyl.

In some embodiments, A is substituted with one R⁴. In some embodiments, A has the structure:

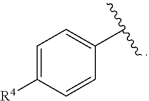

In some embodiments, A is substituted with two R⁴. In some embodiments, at least one R⁴ is C₁₋₆ alkyl. In some embodiments, at least one R⁴ is methyl. In some embodiments, at least one R⁴ is halo. In some embodiments, R⁴ is bromo. In some embodiments, at least one R⁴ is C₁₋₆ alkoxy. In some embodiments, at least one R⁴ is methoxy.

In some embodiments, B is substituted with one R⁵. In some embodiments, B is substituted with two R⁵. In some embodiments, B has the structure

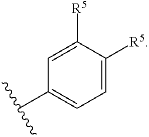

In some embodiments, at least one R⁵ is halo. In some embodiments, at least one R⁵ is fluoro or chloro. In some embodiments, one R⁵ is fluoro and the other R⁵ is chloro. In some embodiments, at least one R⁵ is C₁₋₆ alkoxy. In some embodiments, at least one R⁵ is methoxy. In some embodiments, one R⁵ is halo and the other R⁵ is C₁₋₆ alkoxy. In some embodiments, one R⁵ is chloro and the other R⁵ is methoxy.

In some embodiments, each R⁴ and R⁵ is independently C₁₋₆ alkyl, halo, or C₁₋₆ alkoxy. In some embodiments, R⁶ is C₁₋₆ alkyl, (C═O)R³, (C═O)OR³, or CON(R³)₂.

In various embodiments, X is O—C₀₋₆alkylene or S—C₀₋₆alkylene. In some embodiments, X is S—C₀₋₆alkylene. In some embodiments, X is O, S, O—CH₂—, or S—CH₂—. In various embodiments, Y is C₀₋₂alkylene. In some embodiments, Y is null or CH₂. In some embodiments, X is NR³—CH₂, O—CH₂—, or S—CH₂—, and Y is null. In some embodiments, X is NR³—CH₂, O—CH₂—, or S—CH₂—, and Y is CH₂. In some embodiments, R³ is H.

In various embodiments, X is C₁₋₆alkylene. In some embodiments, X is C₂₋₆alkenylene or C₂₋₆alkynylene. In various embodiments, Y is C₀₋₂alkylene. In some embodiments, Y is null (a bond) or CH₂. In various embodiments, Y is C₃₋₆ alkenylene or C₃₋₆ alkynylene.

As used herein, reference to an element, whether by description or chemical structure, encompasses all isotopes of that element unless otherwise described. By way of example, the term “hydrogen” or “H” in a chemical structure as used herein is understood to encompass, for example, not only ¹H, but also deuterium (²H), tritium (³H), and mixtures thereof unless otherwise denoted by use of a specific isotope. Other specific non-limiting examples of elements for which isotopes are encompassed include carbon, phosphorous, idodine, and fluorine.

It is understood that, in any compound disclosed herein having one or more chiral centers, if an absolute stereochemistry is not expressly indicated, then each center may independently be of R-configuration or S-configuration or a mixture thereof. Thus, the compounds provided herein may be enantiomerically pure or be stereoisomeric mixtures.

Further, compounds provided herein may be scalemic mixtures. Moreover, in any compound disclosed herein having more than one chiral center, then all diastereomers of that compound are embraced. In addition, it is understood that in any compound having one or more double bond(s) generating geometrical isomers that can be defined as E or Z each double bond may independently be E or Z or a mixture thereof. Likewise, all tautomeric forms are also intended to be included.

### Definitions

As used herein, the term “alkyl” refers to straight chained and branched saturated hydrocarbon groups containing one to thirty carbon atoms, for example, one to twenty carbon atoms, or one to ten carbon atoms. The term Cn means the alkyl group has “n” carbon atoms. For example, C₄ alkyl refers to an alkyl group that has 4 carbon atoms. C₁-C₇ alkyl refers to an alkyl group having a number of carbon atoms encompassing the entire range (e.g., 1 to 7 carbon atoms), as well as all subgroups (e.g., 1-6, 2-7, 1-5, 3-6, 1, 2, 3, 4, 5, 6, and 7 carbon atoms). Nonlimiting examples of alkyl groups include, methyl, ethyl, n-propyl, isopropyl, n-butyl, sec-butyl (2-methylpropyl), t-butyl (1,1-dimethylethyl), 3,3-dimethylpentyl, and 2-ethylhexyl. Unless otherwise indicated, an alkyl group can be an unsubstituted alkyl group or a substituted alkyl group.

The term “alkylene” used herein refers to an alkyl group having a substituent. For example, an alkylene group can be —CH₂CH₂— or —CH₂—. The term Cn means the alkylene group has “n” carbon atoms. For example, C₁₋₆ alkylene refers to an alkylene group having a number of carbon atoms encompassing the entire range, as well as all subgroups, as previously described for “alkyl” groups. Unless otherwise indicated, an alkylene group can be an unsubstituted alkylene group or a substituted alkylene group. “Alkenylene” and “alkynylene” are similarly defined, but for alkene or alkyne groups.

As used herein, the term “cycloalkyl” refers to a cyclic hydrocarbon group containing three to eight carbon atoms (e.g., 3, 4, 5, 6, 7, or 8 carbon atoms). The term Cn means the cycloalkyl group has “n” carbon atoms. For example, C5 cycloalkyl refers to a cycloalkyl group that has 5 carbon atoms in the ring. C₆-C₈ cycloalkyl refers to cycloalkyl groups having a number of carbon atoms encompassing the entire range (e.g., 6 to 8 carbon atoms), as well as all subgroups (e.g., 6-7, 7-8, 6, 7, and 8carbon atoms). Nonlimiting examples of cycloalkyl groups include cyclopropyl, cyclobutyl, cyclopentyl, cyclohexyl, cycloheptyl, and cyclooctyl. Unless otherwise indicated, a cycloalkyl group can be an unsubstituted cycloalkyl group or a substituted cycloalkyl group. The cycloalkyl groups described herein can be isolated or fused to another cycloalkyl group, a heterocycle group, an aryl group and/or a heteroaryl group. When a cycloalkyl group is fused to another cycloalkyl group, then each of the cycloalkyl groups can contain three to eight carbon atoms unless specified otherwise. Unless otherwise indicated, a cycloalkyl group can be unsubstituted or substituted.

As used herein, the term “heterocycle” is defined similarly as cycloalkyl, except the ring contains one to three heteroatoms independently selected from oxygen, nitrogen, and sulfur. In particular, the term “heterocycle” refers to a monocyclic ring or fused bicyclic ring containing a total of three to twelve atoms (e.g., 3-8, 5-8, 3-6, 3, 4, 5, 6, 7, 8, 9, 10, 11, or 12), of which 1, 2, or 3 of the ring atoms are heteroatoms independently selected from the group consisting of oxygen, nitrogen, and sulfur, and the remaining atoms in the ring are carbon atoms. Nonlimiting examples of heterocycle groups include piperdine, pyrazolidine, tetrahydrofuran, tetrahydropyran, dihydrofuran, morpholine, and the like. The heterocycle groups described herein can be isolated or fused to a cycloalkyl group, an aryl group, and/or a heteroaryl group. Unless otherwise indicated, a heterocycle group can be unsubstituted or substituted.

Cycloalkyl and heterocycle groups are non-aromatic but can be partially unsaturated ring; and can be optionally substituted with, for example, one to five or one to three groups, independently selected alkyl, alkyleneOH, C(O)NH₂, NH₂, oxo (═O), aryl, alkylenehalo, halo, and OH. Heterocycle groups optionally can be further N-substituted with alkyl (e.g., methyl or ethyl), alkylene-OH, alkylenearyl, and alkyleneheteroaryl. Other substitutions for specific heterocycles and cycloalkyl groups are described herein.

As used herein, the term “aryl” refers to a monocyclic or bicyclic aromatic group, having 6 to 10 ring atoms. Unless otherwise indicated, an aryl group can be unsubstituted or substituted with one or more, and in particular one to five, or one to four or one to three, groups independently selected from, for example, halo, alkyl, alkenyl, OCF₃, NO₂, CN, NC, OH, alkoxy, amino, CO₂H, CO₂alkyl, aryl, and heteroaryl. Aryl groups can be isolated (e.g., phenyl) or fused to a cycloalkyl group (e.g. tetraydronaphthyl), a heterocycle group, and/or a heteroaryl group.

As used herein, the term “heteroaryl” refers to a monocyclic or bicyclic aromatic ring having 5 to 10 total ring atoms, and containing one to four heteroatoms selected from nitrogen, oxygen, and sulfur atom in the aromatic ring. Unless otherwise indicated, a heteroaryl group can be unsubstituted or substituted with one or more, and in particular one to four, substituents selected from, for example, halo, alkyl, alkenyl, OCF₃, NO₂, CN, NC, OH, alkoxy, amino, CO₂H, CO₂alkyl, aryl, and heteroaryl. In some cases, the heteroaryl group is substituted with one or more of alkyl and alkoxy groups. Examples of heteroaryl groups include, but are not limited to, thienyl, furyl, pyridyl, pyrrolyl, oxazolyl, triazinyl, triazolyl, isothiazolyl, isoxazolyl, imidazolyl, pyrazinyl, pyrimidinyl, thiazolyl, and thiadiazolyl.

As used herein, the term “alkoxy” or “alkoxyl” as used herein refers to a “—O-alkyl” group. The alkoxy or alkoxyl group can be unsubstituted or substituted.

As used herein, “halo” refers to F, Cl, I, or Br.

As used herein, the term “therapeutically effective amount” means an amount of a compound or combination of therapeutically active compounds that ameliorates, attenuates or eliminates one or more symptoms of a particular disease or condition (e.g., cancer), or prevents or delays the onset of one of more symptoms of a particular disease or condition.

As used herein, the terms “patient” and “subject” may be used interchangeably and mean animals, such as dogs, cats, cows, horses, and sheep (e.g., non-human animals) and humans. Particular patients or subjects are mammals (e.g., humans).

As used herein, the term “pharmaceutically acceptable” means that the referenced substance, such as a compound of the present disclosure, or a formulation containing the compound, or a particular excipient, are safe and suitable for administration to a patient or subject. The term “pharmaceutically acceptable excipient” refers to a medium that does not interfere with the effectiveness of the biological activity of the active ingredient(s) and is not toxic to the host to which it is administered.

The compounds disclosed herein can be as a pharmaceutically acceptable salt. As used herein, the term “pharmaceutically acceptable salt” refers to those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, S. M. Berge et al. describe pharmaceutically acceptable salts in detail in J. Pharmaceutical Sciences, 1977, 66, 1-19, which is incorporated herein by reference. Pharmaceutically acceptable salts of the compounds of this invention include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, trifluoroacetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, glutamate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Salts of compounds containing a carboxylic acid or other acidic functional group can be prepared by reacting with a suitable base. Such salts include, but are not limited to, alkali metal, alkaline earth metal, aluminum salts, ammonium, N⁺(C₁₋₄alkyl)₄ salts, and salts of organic bases such as trimethylamine, triethylamine, morpholine, pyridine, piperidine, picoline, dicyclohexylamine, N,N [symbol] dibenzylethylenediamine, 2-hydroxyethylamine, bis-(2-hydroxyethyl)amine, tri-(2-hydroxyethyl)amine, procaine, dibenzylpiperidine, dehydroabietylamine, N,N [symbol] bisdehydroabietylamine, glucamine, N-methylglucamine, collidine, quinine, quinoline, and basic amino acids such as lysine and arginine. This invention also envisions the quaternization of any basic nitrogen-containing groups of the compounds disclosed herein. Water or oil-soluble or dispersible products may be obtained by such quaternization. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, lower alkyl sulfonate and aryl sulfonate.

As used herein the terms “treating”, “treat” or “treatment” and the like include preventative (e.g., prophylactic) and palliative treatment.

As used herein, the term “excipient” means any pharmaceutically acceptable additive, carrier, diluent, adjuvant, or other ingredient, other than the active pharmaceutical ingredient (API).

### Synthesis of Compounds of the Disclosure

The compounds disclosed herein can be prepared in a variety of ways using commercially available starting materials, compounds known in the literature, or from readily prepared intermediates, by employing standard synthetic methods and procedures either known to those skilled in the art, or in light of the teachings herein. The synthesis of the compounds disclosed herein can be achieved by generally following the synthetic schemes as described in the Examples section, with modification for specific desired substituents.

Standard synthetic methods and procedures for the preparation of organic molecules and functional group transformations and manipulations can be obtained from the relevant scientific literature or from standard textbooks in the field. Although not limited to any one or several sources, classic texts such as Smith, M. B., March, J., March□s Advanced Organic Chemistry: Reactions, Mechanisms, and Structure, 5^{th} edition, John Wiley & Sons: New York, 2001; and Greene, T. W., Wuts, P. G. M., Protective Groups in Organic Synthesis, 3^{rd} edition, John Wiley & Sons: New York, 1999, are useful and recognized reference textbooks of organic synthesis known to those in the art. The following descriptions of synthetic methods are designed to illustrate, but not to limit, general procedures for the preparation of compounds of the present disclosure.

The synthetic processes disclosed herein can tolerate a wide variety of functional groups; therefore, various substituted starting materials can be used. The processes generally provide the desired final compound at or near the end of the overall process, although it may be desirable in certain instances to further convert the compound to a pharmaceutically acceptable salt thereof.

In general, compounds of Formula (1) can be synthesized according to Scheme 1.

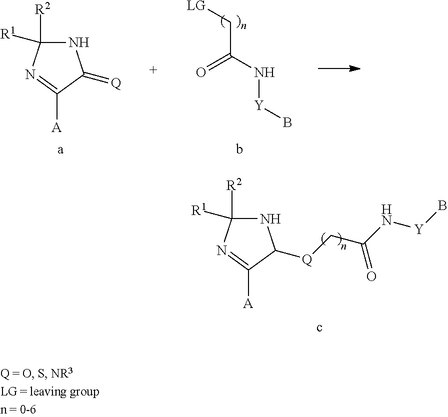

Compounds having structure c can be synthesized using the procedure shown in Scheme 1. Reaction of a substituted 2,5-dihydroimidazole derivative a with an amide compound b in an appropriate solvent e.g., acetonitrile, produces compounds as described herein, i.e., compounds of Formula (1) having structure c. The amide compound b comprises an appropriate leaving group LG chosen based on the nature of group Q, e.g., a halogen or tosylate. Appropriate further derivatization reactions of compounds having structure c can be selected based on the nature of substituents R¹, R², A, Y, and B.

The coupling of compounds a and b can be catalyzed by appropriate reagents selected based on the precise nature of compounds a and b. For example, when the LG of compound b is a halogen (e.g., when LG is chloro), the coupling of compounds a and b can be catalyzed by a base e.g., sodium carbonate or potassium carbonate. Occasionally, the coupling reaction may not require a catalyst.

Occasionally, before coupling with a compound having structure b, a compound a having Q selected from O, S, and NR³ can be transformed into a compound having Q selected from a different member of the group consisting of O, S, and NR³ by treatment with an appropriate reagent. For example, a compound having a structure a with Q=O can be transformed into a compound having a structure a with Q=S by treatment with a thiation reagent, e.g., Lawesson's reagent or phosphorus pentasulfide. Such a compound can then be coupled with a compound having structure b to produce a compound described herein, i.e., a compound of Formula (I) having structure c.

Compounds a and b can be purchased commercially or prepared by a variety of methods from commercially-available starting materials. For example, amide compounds having structure b can be prepared by the reaction of e.g. an acyl chloride with an amine.

Further derivatization reactions to transform compounds having structure c into other compounds disclosed herein can be selected based on the nature of the substituents R¹, R², A, Y, and B in compound c and the functionality desired in the derivative compound.

For example, R¹ and R² together with the carbon atom to which they are attached can form a heterocycling ring, e.g. a piperidine ring, which can be further derivatized by methods known in the art (e.g., methylation, addition of protecting groups, etc.) to form a variety of other compounds of Formula (I) described herein.

### Pharmaceutical Formulations, Dosing, and Routes of Administration

Further provided are pharmaceutical formulations comprising a compound as described herein (e.g., compounds of Formula I, or pharmaceutically acceptable salts thereof) and a pharmaceutically acceptable excipient.

The compounds described herein can be administered to a subject in a therapeutically effective amount (e.g., in an amount sufficient to prevent or relieve the symptoms of a disease or disorder associated with aberrant EGFR, KRAS, BRAF, and/or cMET). The compounds can be administered alone or as part of a pharmaceutically acceptable composition or formulation. In addition, the compounds can be administered all at once, multiple times, or delivered substantially uniformly over a period of time. It is also noted that the dose of the compound can be varied over time.

A particular administration regimen for a particular subject will depend, in part, upon the compound, the amount of compound administered, the route of administration, and the cause and extent of any side effects. The amount of compound administered to a subject (e.g., a mammal, such as a human) in accordance with the disclosure should be sufficient to effect the desired response over a reasonable time frame. Dosage typically depends upon the route, timing, and frequency of administration. Accordingly, the clinician titers the dosage and modifies the route of administration to obtain the optimal therapeutic effect, and conventional range-finding techniques are known to those of ordinary skill in the art.

Purely by way of illustration, the method comprises administering, e.g., from about 0.1 mg/kg up to about 100 mg/kg of a compound as disclosed herein, depending on the factors mentioned above. In other embodiments, the dosage ranges from 1 mg/kg up to about 100 mg/kg; or 5 mg/kg up to about 100 mg/kg; or 10 mg/kg up to about 100 mg/kg. Some conditions require prolonged treatment, which may or may not entail administering lower doses of compound over multiple administrations. If desired, a dose of the compound is administered as two, three, four, five, six or more sub-doses administered separately at appropriate intervals throughout the day, optionally, in unit dosage forms. The treatment period will depend on the particular condition, and may last one day to several months.

Suitable methods of administering a physiologically-acceptable composition, such as a pharmaceutical composition comprising the compounds disclosed herein (e.g., compounds of Formula (I)), are well known in the art. Although more than one route can be used to administer a compound, a particular route can provide a more immediate and more effective reaction than another route. Depending on the circumstances, a pharmaceutical composition comprising the compound is applied or instilled into body cavities, absorbed through the skin or mucous membranes, ingested, inhaled, and/or introduced into circulation. For example, in certain circumstances, it will be desirable to deliver a pharmaceutical composition comprising the agent orally, through injection, or by one of the following means: intravenous, intraperitoneal, intracerebral (intra-parenchymal), intracerebroventricular, intramuscular, intra-ocular, intraarterial, intraportal, intralesional, intramedullary, intrathecal, intraventricular, transdermal, subcutaneous, intraperitoneal, intranasal, enteral, topical, sublingual, urethral, vaginal, or rectal. The compound can be administered by sustained release systems, or by implantation devices.

To facilitate administration, the compound is, in various aspects, formulated into a physiologically-acceptable composition comprising a carrier (e.g., vehicle, adjuvant, or diluent). The particular carrier employed is limited only by chemico-physical considerations, such as solubility and lack of reactivity with the compound, and by the route of administration. Physiologically-acceptable carriers are well known in the art. Illustrative pharmaceutical forms suitable for injectable use include sterile aqueous solutions or dispersions and sterile powders for the extemporaneous preparation of sterile injectable solutions or dispersions (for example, see U.S. Pat. No. 5,466,468). Injectable formulations are further described in, e.g., Pharmaceutics and Pharmacy Practice, J. B. Lippincott Co., Philadelphia. Pa., Banker and Chalmers, eds., pages 238-250 (1982), and ASHP Handbook on Injectable Drugs, Toissel, 4th ed., pages 622-630 (1986)). A pharmaceutical composition comprising the compound is, in one aspect, placed within containers, along with packaging material that provides instructions regarding the use of such pharmaceutical compositions. Generally, such instructions include a tangible expression describing the reagent concentration, as well as, in certain embodiments, relative amounts of excipient ingredients or diluents (e.g., water, saline or PBS) that may be necessary to reconstitute the pharmaceutical composition.

Compositions suitable for parenteral injection may comprise physiologically acceptable sterile aqueous or nonaqueous solutions, dispersions, suspensions, or emulsions, and sterile powders for reconstitution into sterile injectable solutions or dispersions. Examples of suitable aqueous and nonaqueous carriers, diluents, solvents, or vehicles include water, ethanol, polyols (propylene glycol, polyethylene glycol, glycerol, and the like), suitable mixtures thereof, vegetable oils (such as olive oil) and injectable organic esters such as ethyl oleate. Proper fluidity can be maintained, for example, by the use of a coating such as lecithin, by the maintenance of the required particle size in the case of dispersions, and by the use of surfactants.

These compositions may also contain adjuvants such as preserving, wetting, emulsifying, and dispersing agents. Microorganism contamination can be prevented by adding various antibacterial and antifungal agents, for example, parabens, chlorobutanol, phenol, sorbic acid, and the like. It may also be desirable to include isotonic agents, for example, sugars, sodium chloride, and the like. Prolonged absorption of injectable pharmaceutical compositions can be brought about by the use of agents delaying absorption, for example, aluminum monostearate and gelatin.

Solid dosage forms for oral administration include capsules, tablets, powders, and granules. In such solid dosage forms, the active compound is admixed with at least one inert customary excipient (or carrier) such as sodium citrate or dicalcium phosphate or (a) fillers or extenders, as for example, starches, lactose, sucrose, mannitol, and silicic acid; (b) binders, as for example, carboxymethylcellulose, alginates, gelatin, polyvinylpyrrolidone, sucrose, and acacia; (c) humectants, as for example, glycerol; (d) disintegrating agents, as for example, agar-agar, calcium carbonate, potato or tapioca starch, alginic acid, certain complex silicates, and sodium carbonate; (a) solution retarders, as for example, paraffin; (f) absorption accelerators, as for example, quaternary ammonium compounds; (g) wetting agents, as for example, cetyl alcohol and glycerol monostearate; (h) adsorbents, as for example, kaolin and bentonite; and (i) lubricants, as for example, talc, calcium stearate, magnesium stearate, solid polyethylene glycols, sodium lauryl sulfate, or mixtures thereof. In the case of capsules, and tablets, the dosage forms may also comprise buffering agents. Solid compositions of a similar type may also be used as fillers in soft and hard filled gelatin capsules using such excipients as lactose or milk sugar, as well as high molecular weight polyethylene glycols, and the like.

Solid dosage forms such as tablets, dragees, capsules, pills, and granules can be prepared with coatings and shells, such as enteric coatings and others well known in the art. The solid dosage forms may also contain opacifying agents. Further, the solid dosage forms may be embedding compositions, such that they release the active compound or compounds in a certain part of the intestinal tract in a delayed manner. Examples of embedding compositions that can be used are polymeric substances and waxes. The active compound can also be in micro-encapsulated form, optionally with one or more excipients.

Liquid dosage forms for oral administration include pharmaceutically acceptable emulsions, solutions, suspensions, syrups, and elixirs. In addition to the active compounds, the liquid dosage form may contain inert diluents commonly used in the art, such as water or other solvents, solubilizing agents and emulsifiers, as for example, ethyl alcohol, isopropyl alcohol, ethyl carbonate, ethyl acetate, benzyl alcohol, benzyl benzoate, propylene glycol, 1,3-butylene glycol, dimethylformamide, oils, in particular, cottonseed oil, groundnut oil, corn germ oil, olive oil, castor oil, and sesame seed oil, glycerol, tetrahydrofurfuryl alcohol, polyethylene glycols and fatty acid esters of sorbitan, or mixtures of these substances, and the like.

Besides such inert diluents, the composition can also include adjuvants, such as wetting agents, emulsifying and suspending agents, sweetening, flavoring, and perfuming agents. Suspensions, in addition to the active compound, may contain suspending agents, as for example, ethoxylated isostearyl alcohols, polyoxyethylene sorbitol and sorbitan esters, microcrystalline cellulose, aluminum metahydroxide, bentonite, agar-agar, and tragacanth, or mixtures of these substances, and the like.

Upon formulation, solutions will be administered in a manner compatible with the dosage formulation and in such amount as is therapeutically effective. The formulations are easily administered in a variety of dosage forms such as injectable solutions, drug release capsules and the like. For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous and intraperitoneal administration.

In jurisdictions that forbid the patenting of methods that are practiced on the human body, the meaning of “administering” of a composition to a human subject shall be restricted to prescribing a controlled substance that a human subject will self-administer by any technique (e.g., orally, inhalation, topical application, injection, insertion, etc.). The broadest reasonable interpretation that is consistent with laws or regulations defining patentable subject matter is intended. In jurisdictions that do not forbid the patenting of methods that are practiced on the human body, the “administering” of compositions includes both methods practiced on the human body and also the foregoing activities.

### Methods of Use

The compounds described herein can modulate EGFR, KRAS, cMET, and/or BRAF. In some embodiments, the compounds inhibit EGFR dimerization. In various embodiments, the compounds induce EGFR degradation. In various embodiments, the compounds inhibit KRAS. In various embodiments, the compounds inhibit cMET. In various embodiments, the compounds inhibit BRAF.

Although EGFR has clearly been identified as an oncogene and an important molecular target in cancer, there is still a great need and opportunity for an improved approach to modulate the activity of this oncogene. Using a cell penetrating peptide that blocks dimerization (Disruptin) or siRNA, it has been shown that EGFR degradation has a profound effect on cell survival, even in TKI resistant cells.

The approach of degrading EGFR rather than simply inhibiting its kinase activity overcomes the resistance to osimertinib that invariably develops in patients with non-small cell lung cancer. While the focus of this application is on lung cancers, additional and important clinical opportunities also exist in other cancers that are driven by EGFR, such as head & neck, colorectal, and glioblastoma. Targeted selective degradation of an oncoprotein in tumors therefore represents a novel mechanism beyond inhibition of the kinase activity, and this approach might be applicable to other oncogenic proteins.

The compounds disclosed herein are particularly advantageous for the treatment or prevention of diseases or disorders caused by aberrant EGFR activity.

As used herein, “aberrant EGFR activity” refers to activity associated with mutation and overexpression of the epidermal growth factor receptor (EGFR). Such mutation and overexpression is associated with the development of a variety of cancers (Shan et al., Cell 2012, 149(4) 860-870).

Given the importance of the biological roles of EGFR, the compounds of the present disclosures are useful for a number of applications in a variety of settings. For example and most simplistically, the active agents of the present disclosures are useful for inhibiting the dimerization of EGFR in a cell. In this regard, the present disclosures provide a method of inhibiting the dimerization of EGFR in a cell. The method comprises contacting the cell with a compound of the present disclosures, or a pharmaceutically acceptable salt thereof, in an amount effective to inhibit the dimerization. In some aspects, the cell is part of an in vitro or ex vivo cell culture or in vitro or ex vivo tissue sample. In some aspects, the cell is an in vivo cell. In certain embodiments, the method is intended for research purposes, and, in other embodiments, the method is intended for therapeutic purposes.

Inhibition of EGFR dimerization leads to an increase in EGFR degradation. Accordingly, the present disclosures further provides a method of increasing EGFR degradation in a cell. The method comprises contacting the cell with a compound of the present disclosures, or a pharmaceutically acceptable salt thereof, in an amount effective to increase the degradation. In some aspects, the cell is part of an in vitro or ex vivo cell culture or in vitro or ex vivo tissue sample. In some aspects, the cell is an in vivo cell. In certain embodiments, the method is intended for research purposes, and, in other embodiments, the method is intended for therapeutic purposes.

As shown herein, a compound that inhibits dimerization of EGFR increases tumor cell death. Thus, the present disclosures provides a method of increasing tumor cell death in a subject. The method comprises administering to the subject a compound of the present disclosure, or a pharmaceutically acceptable salt thereof, in an amount effective to increase tumor cell death.

In accordance with the foregoing, the present disclosure further provides methods of treating a cancer in a subject comprising administering to the subject a compound of the present disclosure, or a pharmaceutically acceptable salt thereof, in an amount effective to treat the cancer in the subject. In some cases, the cancer is characterized by presence of at least one deleterious KRAS mutation. A deleterious KRAS mutation can be one of the following mutations: G12D, G12V, and G13D. The cancer may also be characterized by the presence of one or more of the following EGFR mutations: L858R, T790M, C797S, S7681, del Exon 19, or a combination thereof.

As used herein, the term “treat,” as well as words related thereto, do not necessarily imply 100% or complete treatment. Rather, there are varying degrees of treatment of which one of ordinary skill in the art recognizes as having a potential benefit or therapeutic effect. In this respect, the methods of treating cancer of the present disclosures can provide any amount or any level of treatment of cancer. Furthermore, the treatment provided by the method of the present disclosures may include treatment of one or more conditions or symptoms of the cancer, being treated. Also, the treatment provided by the methods of the present disclosures may encompass slowing the progression of the cancer. For example, the methods can treat cancer by virtue of reducing tumor or cancer growth, reducing metastasis of tumor cells, increasing cell death of tumor or cancer cells, and the like.

The cancer treatable by the methods disclosed herein may be any cancer, e.g., any malignant growth or tumor caused by abnormal and uncontrolled cell division that may spread to other parts of the body through the lymphatic system or the blood stream. In some embodiments, the cancer is a cancer in which an EGFR is expressed by the cells of the cancer. In some aspects, the cancer is a cancer in which an EGFR protein is over-expressed, the gene encoding EGFR is amplified, and/or an EGFR mutant protein (e.g., truncated EGFR, point-mutated EGFR) is expressed.

The cancer in some aspects is one selected from the group consisting of acute lymphocytic cancer, acute myeloid leukemia, alveolar rhabdomyosarcoma, bone cancer, brain cancer, breast cancer, cancer of the anus, anal canal, or anorectum, cancer of the eye, cancer of the intrahepatic bile duct, cancer of the joints, cancer of the neck, gallbladder, or pleura, cancer of the nose, nasal cavity, or middle ear, cancer of the oral cavity, cancer of the vulva, leukemia (e.g., chronic lymphocytic leukemia), chronic myeloid cancer, colon cancer, esophageal cancer, cervical cancer, gastrointestinal carcinoid tumor, Hodgkin lymphoma, hypopharynx cancer, kidney cancer, larynx cancer, liver cancer, lung cancer, malignant mesothelioma, melanoma, multiple myeloma, nasopharynx cancer, non-Hodgkin lymphoma, ovarian cancer, pancreatic cancer, peritoneum, omentum, and mesentery cancer, pharynx cancer, prostate cancer, rectal cancer, renal cancer (e.g., renal cell carcinoma (RCC)), small intestine cancer, soft tissue cancer, stomach cancer, testicular cancer, thyroid cancer, ureter cancer, and urinary bladder cancer. In particular aspects, the cancer is selected from the group consisting of: head and neck, ovarian, cervical, bladder and oesophageal cancers, pancreatic, gastrointestinal cancer, gastric, breast, endometrial and colorectal cancers, hepatocellular carcinoma, glioblastoma, bladder, lung cancer, e.g., non-small cell lung cancer (NSCLC), bronchioloalveolar carcinoma. In particular aspects, the cancer is an osimertinib-resistant cancer. In some cases, the cancer is pancreatic cancer, head and neck cancer, melanoma, colon cancer, renal cancer, leukemia, or breast cancer. In some cases, the cancer is melanoma, colon cancer, renal cancer, leukemia, or breast cancer. In some cases, the cancer to be treated in a method as disclosed herein can be pancreatic cancer, colorectal cancer, head and neck cancer, lung cancer, e.g., non-small cell lung cancer (NSCLC), ovarian cancer, cervical cancer, gastric cancer, breast cancer, hepatocellular carcinoma, glioblastoma, liver cancer, malignant mesothelioma, melanoma, multiple myeloma, prostate cancer, or renal cancer. In some embodiments, the cancer is pancreatic cancer, colorectal cancer, head and neck cancer, or lung cancer. In some embodiments, the cancer is cetuximab-resistant cancer or osimertinib-resistant cancer.

Uses of the compounds disclosed herein in the preparation of a medicament for modulating EGFR, KRAS, cMET, and/or BRAF, or for treating or preventing a disease or disorder associated with aberrant EGFR, KRAS, cMET, and/or BRAF activity also are provided herein.

In view of the many possible embodiments to which the principles of the disclosure may be applied, it should be recognized that the illustrated embodiments are only examples and should not be taken as limiting the scope of the invention.

### EXAMPLES

Compounds as disclosed herein are synthesized accordingly to synthetic organic techniques known in the art and tested in pharmaceutical assays as disclosed below.

### Pharmacokinetics Studies

Evaluation of Compounds in KRAS Mutant Head and Neck Cancer

Tumor-bearing mice are treated with compound via oral gavage biweekly for one week. The resulting effect of compound on tumor volume is compared to control mice which did not receive the test compound, and control mice which received cetuximab, a known EGFR inhibitor.

Evaluation of Compounds in KRAS Mutant Colorectal and Pancreatic Cancer

Cell Lines: A study is conducted to evaluate the activity of compounds in a KRAS G13D driven cetuximab-resistant colorectal cell-line (HCT-116) using clonogenic survival assays and in a pancreatic cancer cell line (Panc1) that contains KRAS G12D mutation.

Cells are plated at clonal density in 60 or 100 mm culture dishes in triplicate one day before treatment with a range of concentrations (e.g., 0-10 micro M). Eight to twelve days later, cells are fixed with acetic acid/methanol (1:7, v/v), stained with crystal violet (0.5%, w/v), and counted using a stereomicroscope. Drug cytotoxicity (surviving drug-treated cells) are measured and normalized to the survival of the untreated control cells.

The effect of compounds on EGFR, ERK and AKT are also evaluated by immunoblotting. The immunoblotting is performed by following the protocol below:

Cells are plated in 60-mm dishes at a density of 3×10⁵ cells per dishes and incubated overnight or to 70% confluence. The cells are treated with the vehicle (DMSO) or compound and then harvested at various time points. The pellets are washed twice with ice-cold PBS and re-suspended in lysis buffer for 30 min. After sonication, particulate material is removed by centrifugation at 13,000 rpm for 10 min at 4° C. The soluble protein fraction is heated to 95° C. for 5 min and then applied to a 4-12% Bis-Tris precast gel (Invitrogen) and transferred onto a PVDF membrane. Membranes are incubated for 1 hour at room temperature in blocking buffer consisting of 5% BSA and 1% normal goat serum in Tris-buffered saline (137 mM NaCl, 20 mM Tris-HCl (pH 7.6), 0.1% (v/v) Tween 20). Membranes are subsequently incubated overnight at 4° C. with the primary antibody in blocking buffer, washed, and incubated for 1 hour with horseradish peroxidase-conjugated secondary antibody. After three additional washes in Tris-buffered saline, bound antibody is detected by enhanced chemiluminescence plus reagent. For quantification of relative protein levels, immunoblot films are scanned and analyzed using Image J 1.32j software.

The activity of compounds disclosed herein are also tested against 60 different human tumor cell lines at the National Cancer Institute, using the standard NCI 60 screening protocol.

### Viability Assay

The viability of cells upon treatment is assessed by CellTiter-Blue® reagent following the manufacturer's protocol in RKO, UM10B, UM1, MCR5, and UMCC92 cells. Briefly, 10,000 cells are plated in 96-well plate in quadruplets. One day after seeding, cells are treated with a range of concentrations of compound (0.1 to 30 micromolar). 3-days post-treatment, cells are incubated with the CellTiter-Blue® reagent for 4 hr. Only the viable cells convert the redox dye (reszurin) into a fluorescent product (resofurin). The emission of fluorescence (excitation 560 nM) is measured at 590 nM. The IC₅₀ value is calculated as the mean concentration of compounds required to inhibit cell proliferation as measured by the fluorescence at 590 nM by 50 percent compared to the vehicle-treated controls.

### Validation of EGFR Reporter In Vivo

Briefly, once the tumors reach the size of about 100 mm³, mice are imaged to obtain the basal bioluminescence and effect of compound on different time points. The effect of treatment on EGFR protein level is confirmed by immunoblotting after 48 hours of treatment.

### In Vivo Activity of Compound

Nude mice bearing UMSCC74B (˜100 mm²) are treated with (30 mg/kg, daily for one week) or with vehicle (5% DMSO in PBS). Each group has at least 5 mice. Tumor volume and body weight are recorded 3-4 times a week, and change in the average tumor volume with time is plotted.

For the Compound treatment group, day 0 is defined as the first day of treatment. In vehicle control mice, day 0 is defined as the day when the tumor volume was closest to the mean tumor volume in Compound treatment groups on the day of treatment initiation. To assess whether tumor volume growth rates differ by treatment, mixed effect models are fit with random intercept terms at the mouse levels to account for correlated outcomes over time within a tumor and between 2 tumors within a mouse.

### Effect of Compound in an Osimertinib Resistant Tumor Model.

To test the activity of Compound against osimertinib resistant EGFR driven tumors, an ascites tumor model using Ba/F3-AZR cells (L858R+T790M+C797S-EGFR) is used. 5 million, BA/F3-AZR cells are injected via i.p. injection into 6-week old female nude mice. To test the efficacy of Compound compared to osimertinib, injected 15 mice are injected with Ba/F3-AZR cells. 18 days after injection of tumor cells, mice are randomized into three groups. Mice are treated with vehicle, a single oral dose of 30 mg/kg osimertinib, or 30 mg/kg Compound via i.p. injection. The health of mice is monitored and mice are euthanized according to ULAM end-stage guidelines.

### Preliminary Safety Tests of Compound in a Mouse Model.

A preliminary test on the safety of a daily dose of 30 mg/kg for one week of compound is performed using C57BL6 mice. The overall health and weight of a group of 6 mice is monitored during treatment.

### NCI 60 Cell Line Screen

The activity of Compound is tested against 60 different human tumor cell lines at the National Cancer Institute, using the standard NCI 60 screening protocol, as shown in the below table.

TABLE

| Panel | Cell Line |
|---|---|
| Melanoma | SK-MEL-5 |
| Colon Cancer | HCT-116 |
| Melanoma | M14 |
| Renal Cancer | 786-0 |
| Melanoma | UACC-62 |
| Melanoma | LOX IMVI |
| Colon Cancer | COLO 205 |
| Melanoma | MALME-3M |
| Melanoma | SK-MEL-28 |
| Colon Cancer | HT29 |
| Leukemia | K-562 |
| Melanoma | UACC-257 |
| Colon Cancer | HCC-2998 |
| Breast Cancer | MDA-MB-468 |
| Breast Cancer | MCF7 |
| Leukemia | HL-60(TB) |
| Breast Cancer | MDA-MB-231/ATCC |

### Effect of Compound in a Pancreatic Tumor Model

6-week old KC mice are treated with Compound via oral gavage (30 mg/kg body weight, daily). The resulting effect on Panln levels are observed compared to control mice which did not receive Compound.

### Effect of Compound in a Head and Neck Tumor Model

Mouse xenographs of UMSCC74B, a head and neck tumor cell line, are treated with Compound via oral gavage (30 mg/kg body weight, twice weekly). The resulting effect on tumor volume is observed compared to control mice which did not receive Compound, and control mice which received cetuximab.

What is claimed:

1. A compound, or pharmaceutically acceptable salt thereof, having a structure of Formula I: 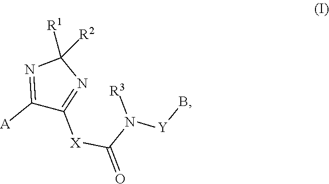
wherein
X is C₁₋₆ alkylene, C₂₋₆ alkenylene, C₂₋₆ alkynylene, C₃₋₁₀ cycloalkylene, 4-6 membered heterocycle, O—C₀₋₆alkylene, O—C₂₋₆ alkenylene, O—C₂₋₆ alkynylene, O—C₃₋₁₀ cycloalkylene, O-(4-6 membered heterocyclene), S—C₀₋₆alkylene, S—C₂₋₆ alkenylene, S—C₂₋₆ alkynylene, S—C₃₋₁₀ cycloalkylene, S-(4-6 membered heterocyclene), NR³—C₀₋₆alkylene, NR³—C₂₋₆ alkenylene, NR³—C₂₋₆ alkynylene, NR³—C₃₋₁₀ cycloalkylene, or NR³-(4-6 membered heterocyclene), and X is optionally substituted with 1-5 groups independently selected from R³;
Y is C₀₋₆alkylene, C₃₋₆alkenylene, or C₃₋₆alkynylene, and Y is optionally substituted with 1-3 groups independently selected from halo, N(R³)₂, and R³;
A is C₆₋₁₀ aryl or 5-10 membered heteroaryl having 1-4 heteroatoms selected from N, O, and S, and A is optionally substituted with 1 to 3 R⁴;
B is C₆₋₁₀ aryl, 5-10 membered heteroaryl having 1-4 heteroatoms selected from N, O, and S, 3-8 membered cycloalkyl ring, or a 4-10 membered heterocycle having 1-3 heteroatoms selected from N, O, and S, and B is optionally substituted with 1 to 3 R⁵;
R¹ and R² are each independently C₁₋₆ alkyl, C₃₋₆ alkenyl, C₃₋₆ alkynyl, or C₃₋₆ cycloalkyl, or R¹ and R² together with the carbon atom to which they are attached form a 4-8 membered cycloalkyl or heterocycle, wherein the heterocycle has 1 or 2 ring heteroatoms selected from O, S, and N, and wherein said cycloalkyl or heterocycle is optionally substituted with 1-2 R⁴; each R³ is independently OH, C₁₋₆ alkyl, C₂₋₆alkenyl, C₂₋₆alkynyl, C₁₋₆alkoxy, phenyl, O-phenyl, benzyl, O-benzyl, C₃₋₆cycloalkyl, 4-10 membered heterocycle having 1 to 4 heteroatoms selected from N, O, and S, or (O)₀₋₁-5-10 membered heteroaryl having 1 to 3 heteroatoms selected from N, O, and S, or two R³ taken together with the atom(s) to which they are attached form a C₃₋₆ cycloalkyl (e.g., C₄₋₆ cycloalkenyl), or 4-6 membered heterocycle having one heteroatom selected from N, O and S;
each R⁴ and R⁵ is independently halo, NO₂, oxo, cyano, C₁₋₄ alkyl, C₁₋₄haloalkyl (e.g., CF₃, CHF₂), C₁₋₄alkoxy, C₁₋₄haloalkoxy (e.g., OCF₃, OCHF₂), C₁₋₄thioalkoxy, C₂₋₄alkenyl, C₂₋₄alkynyl, CHO, C(═O)Rs, C(═O)N(R⁶)₂, S(O)₀₋₂R⁶, SO₂N(R⁶)₂, NH₂, NHR⁶, N(R⁶)₂, NR⁷COR⁶, NR⁷SO₂R⁶, P(═O)(R⁶)₂, C₃₋₆cycloalkyl, 4-10 membered heterocycle having 1 to 4 heteroatoms selected from N, O, and S (e.g., oxetanyl, oxetanyloxy, oxetanylamino, oxolanyl, oxolanyloxy, oxolanylamino, oxanyl oxanyloxy, oxanylamino, oxepanyl, oxepanyloxy, oxepanylamino, azetidinyl, azetidinyloxy, azetidylamino, pyrrolidinyl, pyrolidinyloxy, pyrrolidinylamino, piperidinyl, piperidinyloxy, piperidinylamino, azepanyl, azepanyloxy, azepanylamino, dioxolanyl, dioxanyl, morpholino, thiomorpholino, thiomorpholino-S,S-dioxide, piperazinyl, dioxepanyl, dioxepanyloxy, dioxepanylamino, oxazepanyl, oxazepanyloxy, oxazepanylamino, diazepanyl, diazepanyloxy, or diazepanylamino);
each R⁶ is independently H, C₁₋₆ alkyl, C₁₋₆ haloalkyl, C₃₋₆ alkenyl, C₃₋₆ alkynyl, COOR⁷, CON(R⁷)₂, C₀₋₃alkylene-C₃₋₈cycloalkyl, C₀₋₃alkylene-C₆₋₁₀aryl, C₀₋₃alkylene-(4-10 membered heterocycle having 1-4 heteroatoms selected from N, O, and S), or C₀₋₃alkylene-(5-10 membered heteroaryl having 1-4 heteroatoms selected from N, O, and S), wherein the aryl, heterocyle, or heteroaryl is optionally substituted with 1 to 3 R⁷; and
each R⁷ is independently H, C₁₋₆ alkyl, C₁₋₆ haloalkyl, C₃₋₆ alkenyl, C₃₋₆ alkynyl, C₁₋₄alkoxy, or C₁₋₄haloalkoxy,
with the proviso that the compound is not a compound (or salt thereof) as disclosed in WO 2019/165358.

2. The compound or salt of claim 1, wherein R¹ and R² are each independently C₁₋₆ alkyl.

3. The compound or salt of claim 2, wherein R¹ and R² are each methyl.

4. The compound or salt of claim 1, wherein R¹ and R² together with the carbon atom to which they are attached form a 4-8 membered cycloalkyl or heterocycle.

5. The compound or salt of claim 4, wherein R¹ and R² together with the carbon atom to which they are attached form a 5 or 6 membered cycloalkyl or heterocycle.

6. The compound or salt of claim 5, wherein R¹ and R² together with the carbon atom to which they are attached form a cyclohexyl ring.

7. The compound or salt of claim 5, wherein R¹ and R² together with the carbon atom to which they are attached form a heterocycle having the structure: 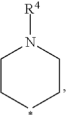
where * indicates the point of attachment to the rest of the compound of Formula I.

8. The compound or salt of any one of claims 1 to 7, wherein A is C₆₋₁₀ aryl.

9. The compound or salt of claim 8, wherein A is phenyl.

10. The compound or salt of any one of claims 1 to 9, wherein B is C₆₋₁₀ aryl.

11. The compound or salt of claim 10, wherein B is phenyl.

12. The compound or salt of any one of claims 1 to 9, wherein B is 5-10 membered heteroaryl having 1-4 heteroatoms selected from N, O, and S.

13. The compound or salt of claim 12, wherein B is pyridinyl.

14. The compound or salt of claim 12, wherein B is quinolinyl.

15. The compound or salt of any one of claims 1 to 9, wherein B is 3-8 membered cycloalkyl.

16. The compound or salt of claim 15, wherein B is 5 or 6 membered cycloalkyl.

17. The compound or salt of any one of claims 1 to 9, wherein B is 3-12 membered heterocycle having 1-3 ring heteroatoms selected from O, S, and N.

18. The compound or salt of any one of claims 1 to 17, wherein A is substituted with one R⁴.

19. The compound or salt of claim 18, wherein A has the structure:

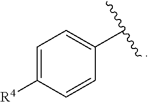

20. The compound or salt of any one of claims 1 to 17, wherein A is substituted with two R⁴.

21. The compound or salt of any one of claims 1 to 20, wherein at least one R⁴ is C₁₋₆ alkyl.

22. The compound or salt of claim 21, wherein is at least one R⁴ is methyl.

23. The compound or salt of any one of claims 1 to 22, wherein at least one R⁴ is halo.

24. The compound or salt of claim 23, wherein R⁴ is bromo.

25. The compound or salt of claim 23 or 24, wherein R⁴ is chloro.

26. The compound or salt of claim 23, 24, or 25, wherein R⁴ is fluoro.

27. The compound or salt of any one of claims 1 to 26, wherein at least one R⁴ is C₁₋₆ alkoxy.

28. The compound or salt of claim 27, wherein at least one R⁴ is methoxy.

29. The compound or salt of any one of claims 1 to 28, wherein B is substituted with one R⁵.

30. The compound or salt of any one of claims 1 to 28, wherein B is substituted with two R⁵.

31. The compound of claim 30, wherein B has the structure

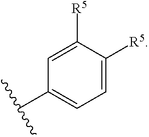

32. The compound or salt of any one of claims 1 to 31, wherein at least one R⁵ is halo.

33. The compound or salt of claim 32, wherein at least one R⁵ is fluoro or chloro.

34. The compound or salt of claim 30 or 32, wherein one R⁵ is fluoro and the other R⁵ is chloro.

35. The compound or salt of any one of claims 1 to 34, wherein at least one R⁵ is C₁₋₆ alkoxy.

36. The compound or salt of claim 35, wherein at least one R⁵ is methoxy.

37. The compound or salt of any one of claims 30 to 36, wherein one R⁵ is halo and the other R⁵ is C₁₋₆ alkoxy.

38. The compound or salt of claim 37, wherein one R⁵ is chloro and the other R⁵ is methoxy.

39. The compound or salt of any one of claims 1 to 38, wherein X is C₁₋₆alkylene.

40. The compound or salt of any one of claims 1 to 38, wherein X is C₂₋₆alkenylene or C₂₋₆alkynylene.

41. The compound or salt of any one of claims 1 to 38, wherein X is C₃₋₁₀ cycloalkylene, or 4-6 membered heterocyclene.

42. The compound or salt of any one of claims 1 to 38, wherein X is O—C₀₋₆alkylene or S—C₀₋₆alkylene.

43. The compound or salt of claim 42, wherein X is O, S, O—CH₂—, or S—CH₂—.

44. The compound or salt of any one of claims 1 to 43, wherein Y is a bond or CH₂.

45. The compound or salt of any one of claims 1 to 43, wherein Y is C₁₋₆alkylene.

46. The compound or salt of any one of claims 1 to 43, wherein Y is C₂₋₆alkenylene or C₂₋₆alkynylene.

47. The compound or salt of any one of claims 1 to 46, wherein R³ is H.

48. A pharmaceutical composition comprising the compound or salt of any one of claims 1 to 47 and a pharmaceutically acceptable carrier or excipient.

49. A method of modulating EGFR comprising administering to a subject in need thereof a therapeutically effective amount of the compound or salt of any one of claims 1 to 47.

50. A method of modulating KRAS comprising administering to a subject in need thereof a therapeutically effective amount of the compound or salt of any one of claims 1 to 47.

51. A method of modulating cMET comprising administering to a subject in need thereof a therapeutically effective amount of the compound or salt of any one of claims 1 to 47.

52. A method of modulating BRAF comprising administering to a subject in need thereof a therapeutically effective amount of the compound or salt of any one of claims 1 to 47.

53. A method of treating cancer in a subject who suffers therefrom, comprising administering to the subject a therapeutically effective amount of the compound or salt of any one of claims 1 to 47.

54. The method of claim 53, wherein the cancer is acute lymphocytic cancer, acute myeloid leukemia, alveolar rhabdomyosarcoma, bone cancer, brain cancer, breast cancer, cancer of the anus, anal canal, or anorectum, cancer of the eye, cancer of the intrahepatic bile duct, cancer of the joints, cancer of the neck, gallbladder, or pleura, cancer of the nose, nasal cavity, or middle ear, cancer of the oral cavity, cancer of the vulva, leukemia (e.g., chronic lymphocytic leukemia), chronic myeloid cancer, colon cancer, esophageal cancer, cervical cancer, gastrointestinal carcinoid tumor, Hodgkin lymphoma, hypopharynx cancer, kidney cancer, larynx cancer, liver cancer, lung cancer, malignant mesothelioma, melanoma, multiple myeloma, nasopharynx cancer, non-Hodgkin lymphoma, ovarian cancer, pancreatic cancer, peritoneum, omentum, and mesentery cancer, pharynx cancer, prostate cancer, rectal cancer, renal cancer (e.g., renal cell carcinoma (RCC)), small intestine cancer, soft tissue cancer, stomach cancer, testicular cancer, thyroid cancer, ureter cancer, or urinary bladder cancer.

55. The method of claim 53, wherein the cancer is selected from lung cancer, colorectal cancer, glioblastoma, and head and neck cancer.

56. The method of claim 53, wherein the cancer is melanoma, colon cancer, renal cancer, leukemia, or breast cancer.

57. The method of any one of claims 53 to 56, wherein the cancer is osimertinib-resistant cancer.

58. The method of any one of claims 53 to 57, wherein the subject is human.
